(19) United States
(12) Patent Application Publication
(10) Pub. No.: US 2024/0352425 A1
(43) Pub. Date: Oct. 24, 2024

(54) METHODS FOR OPTIMIZING REPRODUCTIVE TISSUE DERIVED CELL YIELD AND VIABILITY FOR CLINICAL APPLICATIONS

(71) Applicant: GALLANT PET. INC., San Diego, CA (US)

(72) Inventors: Theodore T. Sand, Poway, CA (US); Linda Black, Carlsbad, CA (US); Samuel Barillas, San Diego, CA (US)

(21) Appl. No.: 18/262,069

(22) PCT Filed: Jan. 18, 2022
(86) PCT No.: PCT/US2022/070219

§ 371 Date: Jul. 19, 2023

Related U.S. Application Data
(60) Provisional application No. 63/139,031, filed on Jan. 19, 2021.

(51) Int. Cl. C12N 5/071

(52) U.S. Cl. CPC C12N 5/0682; C12N 2509/00; C12N 2509/10

(57) ABSTRACT

Disclosed herein are novel methods of processing reproductive tissue, from e.g., spay and neuter procedures, to optimize cell yield and viability in a manner suitable for obtaining multiple therapeutic doses typically required for clinical applications. For example, disclosed herein are novel methods of digesting, agitating and incubating reproductive tissues and fractions thereof, including, for example, reciprocal and/or vibrational mechanical agitation and co-culture with cell suspensions and partially digested tissue to enhance cell migration.

### CROSS-REFERENCE TO RELATED APPLICATIONS

This application claims the benefit of priority of U.S. Provisional Patent Application No. 63/139,031, filed Jan. 19, 2021, which is hereby expressly incorporated by reference in its entirety.

### FIELD OF THE INVENTION

Aspects of the present disclosure relate generally to methods of processing reproductive tissues to optimize cell yield and viability.

### BACKGROUND OF THE INVENTION

Pets and farm animals, like humans, suffer from numerous diseases and disorders as they age. Administration of cellular compositions for therapeutic purposes in the veterinary setting has become increasingly prevalent and promising. However, conventional methods used to process veterinary samples result in significant variability in yield. The ability to reliably generate multiple clinical doses from a minimal number of sources for delivery to patients when needed is critical. Aspects of the present disclosure are addressed to these needs.

### SUMMARY OF THE INVENTION

Disclosed herein are novel methods of processing reproductive tissue, from e.g., spay and neuter procedures, to optimize cell yield and viability, such that multiple therapeutic doses of reproductive tissue derived cells required for clinical applications can be obtained. For example, disclosed herein are novel methods of digesting, agitating and incubating reproductive tissues and fractions thereof, including, for example, reciprocal and/or vibrational mechanical agitation and co-culture with cell suspensions and partially digested tissue to enhance cell migration.

The present invention discloses methods of processing reproductive tissues to optimize cell yield and viability. Embodiments of the present disclosure, as well as features and advantages thereof, will be apparent from the descriptions herein.

Specifically, the present invention relates to a method of processing reproductive tissue to obtain reproductive tissue derived cells comprising: obtaining reproductive tissues such as from spay or neuter procedures; mincing said reproductive tissue; digesting said minced reproductive tissue with an enzymatic solution to obtain a tissue suspension; agitating said tissue suspension such that reciprocal mechanical agitation is applied to the tissue suspension; filtering the tissue suspension into a cell suspension fraction and a partially digested reproductive tissue fraction; centrifuging the cell suspension fraction to form a first centrifuged cell pellet and resuspending the first centrifuged cell pellet into a single cell suspension; incubating the partially digested reproductive tissue fraction in an incubation medium such that cells migrate from the partially digested reproductive tissue fraction into the incubation medium; centrifuging the incubation medium to form a second centrifuged cell pellet and resuspending the second centrifuged cell pellet into a migrating cell suspension; combining the single cell suspension and the migrating cell suspension to obtain the reproductive tissue derived cells.

The reproductive tissues encompassed by the present invention includes tissue that is tissue from the testes, vas deferens, outer epididymis, or any combination thereof, for males and ovaries, fallopian tubes, or uterus, or any combination thereof, for females. The reproductive tissue encompassed by the present invention includes tissue from canines or felines. In some embodiments, the reproductive tissue is isolated from a spay or neuter procedure.

In certain embodiments, the methods of the present invention utilize an enzymatic solution for digestion of the reproductive tissue. In some embodiments, the enzymatic solution is comprised of collagenase or a neutral protease, or both. In certain embodiments, the reproductive tissue is agitated such that the agitating is performed in a dry heat incubator set at 37° C. In some embodiments, the agitating is performed for at least 40, 50, 60, 70, 80, 90, 100, 110, 120, 130, 140, 150, 160, 170, 180, 190, or 200 minutes, or any time within a range defined by any two of the aforementioned times. In certain embodiments, the agitating is performed for at least forty minutes or more. In some embodiments, the agitating is performed for a maximum of 5, 10, 15, 20, 25, 30, 35, or 40 minutes, or any time within a range defined by any two of the aforementioned times. In other embodiments, the agitating is performed for a maximum of forty minutes.

In particular embodiments, the tissue suspension is agitated such that both reciprocal and vibrational mechanical agitation is applied to the tissue suspension. In yet other embodiments, the tissue suspension is agitated such that rotational mechanical agitation is not applied to the tissue suspension. In certain embodiments, the agitation is performed on a platform.

In embodiments with filtration, the filtering step comprises passing the tissue suspension through one or more cell strainers to separate the single cell suspension fraction and the partially digested tissue fraction. In some embodiments, the one or more cell strainers comprise a 300 μm, 100 μm, 70 μm, or 40 μm strainer, or any combination thereof. The single cell suspension fraction may be stored at 4° C.

In embodiments where the partially digested reproductive tissue fraction is incubated, incubating the partially digested reproductive tissue fraction is for no more than 1, 2, 3, 4, 5, 6, 7, 8, 9, 10, 11, 12, 13, 14, 15, 16, 17, 18, 19, 20, 21, 22, 23, or 24 hours, or any time within a range defined by any two of the aforementioned times. In some embodiments, the incubation is for two hours or less.

In certain embodiments, the single cell suspension fraction is cryopreserved.

In other embodiments, the partially digested reproductive tissue fraction is cryopreserved.

In some embodiments, the single cell suspension and the partially digested reproductive tissue are cryopreserved separately. In yet other embodiments, the single cell suspension and the partially digested reproductive tissue fraction are cryopreserved together in the same vial.

In certain therapeutic embodiments, the single cell suspension and the partially digested reproductive tissue fraction are thawed. In some embodiments, the methods disclosed herein further comprise thawing the single cell suspension and the partially digested reproductive tissue fraction.

In some embodiments, the methods disclosed herein further comprise incubating the thawed single cell suspension and the thawed partially digested reproductive tissue together. In other embodiments, the thawed single cell suspension and the thawed partially digested reproductive tissue fraction are incubated together in the same tissue culture flask. The reproductive tissue derived cells may migrate from the partially digested reproductive tissue fraction into the incubation medium. The reproductive tissue derived growth factors may also diffuse from the partially digested reproductive tissue fraction into the incubation medium. The incubation medium is centrifuged to obtain a cell pellet comprising reproductive tissue derived cells.

In some embodiments, combining the single cell suspension and the migrating cell suspension to obtain the reproductive tissue derived cells improves the total yield of single cells isolated from the reproductive tissue. In some embodiments, combining the single cell suspension and the migrating cell suspension to obtain the reproductive tissue derived cells improves the total yield of single cells isolated from the reproductive tissue by at least 50%, 100%, 120%, 140%, 160%, 180%, 200%, 220%, 240%, 260%, 280%, 300%, 320%, 340%, 360%, 380%, or 400% as compared to the total yield of single cells isolated in the cell suspension fraction alone, or any percentage within a range defined by any two of the aforementioned percentages. In some embodiments, combining the single cell suspension and the migrating cell suspension to obtain the reproductive tissue derived cells results in a total yield of single cells isolated from the reproductive tissue of about 3×10\hat{ }6, 4×10\hat{ }6, 5×10\hat{ }6, 6×10\hat{ }6, 7×10\hat{ }6, 8×10\hat{ }6, 9×10\hat{ }6, or 10\hat{ }7 cells per gram of reproductive tissue, or any number of cells within a range defined by any two of the aforementioned number of cells.

Also disclosed herein are methods of cryopreserving partially enzymatically-digested tissue. In some embodiments, the methods comprise obtaining tissue, mincing said tissue, mixing said minced tissue with an enzymatic solution to obtain a tissue suspension, agitating said tissue suspension such that mechanical agitation is applied to the tissue suspension, filtering the tissue suspension into a cell suspension fraction and a partially digested reproductive tissue fraction, and cryopreserving the partially digested tissue fraction. In some embodiments, the enzymatic solution comprises one or more dissociation enzymes and/or neutral proteases. In some embodiments, the enzymatic solution comprises collagenase and/or a neutral protease. In some embodiments, the partially digested tissue fraction is cryopreserved without the cell suspension fraction. In some embodiments, the partially digested tissue fraction is cryopreserved together with the cell suspension fraction.

In any of the embodiments provided herein, the patient may be a dog, cat or horse, or other non-human mammal. In some embodiments, the delivery may be autologous. In some embodiments, the delivery may be allogeneic.

### DETAILED DESCRIPTION OF THE INVENTION

Disclosed herein are novel methods of optimizing reproductive tissue processing methods to obtain higher yields of viable regenerative cells, i.e., in amounts suitable for clinical applications. Reference to certain embodiments will be made in this detailed description, and specific language will be used to describe the embodiments. It will be understood that this description is intended to be illustrative. Any alterations and further modifications in the described embodiments, and any further applications of the principles thereof, are contemplated as would normally occur to one skilled in the art to which this disclosure pertains.

### Definitions

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The “individual”, “patient” or “subject” treated as disclosed herein is, in some embodiments, a mammal. The term “mammal” as used herein includes, but is not limited to, humans, non-human animals, including primates, cattle, sheep, goats, pigs, horses, cats, dogs, rabbits, rodents (e.g., rats or mice), monkeys, etc. In some embodiments, the individual, patient, or subject is a household pet or a livestock animal. In some embodiments, the individual, patient, or subject is a cat (feline), dog (canine), or horse. The subject can be a subject “in need of” the methods disclosed herein or can be a subject that is experiencing a disease state and/or is anticipated to experience a disease state, and the methods and compositions of the invention are used for therapeutic and/or prophylactic treatment. A subject can be a patient, which refers to a subject presenting to a medical provider for diagnosis or treatment of a disease. A subject can be afflicted with or is susceptible to a disease or disorder but may or may not display symptoms of the disease or disorder.

Some embodiments disclosed herein relate to selecting a subject or patient in need. In some embodiments, a subject is selected who has a disease, in need of treatment or therapy for a disease, at risk of contracting a disease, previously had a disease, or does not currently have a disease. In some embodiments, a subject is selected who has previously been non-responsive to another therapy.

The terms “function” and “functional” as used herein refer to a biological, enzymatic, or therapeutic function.

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to or at least about 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

The terms “primary cell fraction” or “PCF” refer to the population of cells obtained from the isolation, dissociation, and/or purification steps taken to prepare a viable single cell suspension from the target tissue prior to culturing steps. The PCF population is distinct from the population of cells that are obtained after the one or more culturing steps, as the relative fraction of certain cell types within the population and the properties of said cell types changes over the one or more culturing steps.

The term “mesenchymal stem cell (MSC)” refers to a population of spindle-shaped adherent cells that exhibit the potential to differentiate into osteogenic, chondrogenic, and adipogenic lineages. MSCs may also be referred as “mesenchymal stromal cells”, “mesenchymal progenitor cells (MPC)”, or “stromal cells”. MSCs have been isolated primarily from bone marrow, but are also present in umbilical cord tissue, umbilical cord blood, white adipose tissue, and placenta tissue, among other tissues. Upon culture, MSCs generally express CD105, CD73, CD90 and generally do not express CD14, CD19, CD34, CD45, and HLA-DR (Class II). Their ability to differentiate into multiple cell types, as well as lack of MHC class II, have prompted investigation for use in autologous or allogeneic regenerative cell therapy.

As used herein, “in vivo” is given its ordinary meaning and refers to the performance of a method inside living organisms, usually animals, mammals, including humans, and plants, as opposed to a tissue extract or dead organism.

As used herein, “ex vivo” is given its ordinary meaning and refers to the performance of a method outside a living organism with little alteration of natural conditions.

As used herein, “in vitro” is given its ordinary meaning and refers to the performance of a method outside of biological conditions, e.g., in a petri dish or test tube.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to cells, nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of cell proteins, cell nucleic acids, plasmid DNA, contaminating viruses, proteasomes, cell culture components, process related components, mycoplasma, pyrogens, bacterial endotoxins, and adventitious agents. Purity can be measured using technologies understood in the art including but not limited to electrophoresis. SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein refers to the actual overall amount of the substance, compound, or material relative to the expected overall amount. For example, the yield of the substance, compound, or material may be at least 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

### Reproductive Tissue Derived Cells

The method of the invention results in a reproductive tissue derived viable cell composition that has distinct advantages in the treatment and prevention of injuries, diseases and disorders. The reproductive tissue derived viable cell composition obtained according to the methods of the invention may include isolated or mixed populations of reproductive tissue derived cells, such as one or more regenerative, anti-inflammatory, anti-apoptotic, immune modulatory and trophic cells, including but not limited to mesenchymal stem cells among others, that are removed from their natural tissue environment and concentrated as compared to their original tissue environment. In certain embodiments, the viable cells are present at a concentration approximately 2, 5, 10, 20, 30, 40, 50, 60, 70, 80, 90, or 100 fold greater than their concentration in the tissue from which they were isolated, when initially pelleted, or any fold concentration within a range defined by any two of the aforementioned fold concentrations.

A wide variety of reproductive tissue derived cell types may be used in embodiments of the present disclosure. For example, the reproductive tissue derived cells can be “regenerative cells” which is used in a broad sense and includes any one or more of any of traditional stem cells, mesenchymal stem cells, progenitor cells, mesenchymal progenitor cells, pre-progenitor cells, pericytes, endothelial cells, epithelial cells, germline stem cells, including spermatogonial or oogenic progenitors, and the like. In some embodiments, administration of the reproductive tissue derived cells treat a patient inflicted by a disease, disorder, or condition. For example, the reproductive tissue derived cells of the present invention may reduce the rate of scar formation, or increase the rate of angiogenic vessel formation for wound healing. In some embodiments, the reproductive tissue derived cell compositions may also include other cell types, such as one or more of the following: red blood cells, white blood cells, platelets, neutrophils, monocyte/macrophages, fibroblasts, fibroblast-like cells, lymphocytes, basophils, pericytes, or peripheral neural progenitors among others.

The reproductive tissue derived cells disclosed in the embodiments herein can be from any suitable species of animal, for example a mammal, such as a human, canine (e.g. dog), feline (e.g. cat), equine (e.g. horse), porcine, ovine, caprine, or bovine mammal. The reproductive tissue derived cells disclosed in the embodiments herein can be obtained from any suitable reproductive tissue or organ, including male or female reproductive tissue or organs. These include, by way of example only, cells derived from testicular tissue, ovarian tissue, uterine tissue, the fallopian tubes, the vas deferens or the outer epididymis. For example, for reproductive tissue samples from male subjects during neuter procedures, cells from the testes may be used in certain methods of the invention with or without excising off the vas deferens and outer epididymis. For reproductive tissue samples from female subjects during spay procedures, cells from the uterus or ovaries may be used in certain methods of the present invention with or without excising off the fallopian tubes. In some embodiments, the reproductive tissue derived cells generally can be a cell population expressing markers for both adherent and non-adherent cells. These markers can be detected using conventional methods, such as flow cytometry.

In some embodiments, the reproductive tissue derived viable cell compositions obtained according to certain methods of the invention may further include additional non-cellular tissue components. Such non-cellular components may be soluble factors or insoluble components, such as lipids, or both. Examples of such non-cellular tissue components include but are not limited to extracellular matrix proteins, proteoglycans, secreted factors, cytokines, growth factors, differentiation-inducing factors, differentiation-inhibiting factors, or fragments thereof. In one embodiment, the non-cell component populations include but are not limited to collagen, thrombospondin, fibronectin, vitronectin, laminin, or fragments thereof. In a particular embodiment, the non-cell component populations include collagen or fragments thereof. Collagens include, but are not limited to, Type I. Type III, Type IV collagen or their isoforms, laminin or other certain isoforms, versican, perlecan, or entactin. Type IV collagen and laminin in particular play an important role in both blood vessel and nerve formation. Again, these additional non-cell components frequently will be present in the originally isolated cell population. However, in certain embodiments, such non-cell components may be added prior to culture expansion prior to administration to a patient. In some embodiments, these additional non-cell components are removed from the cell compositions prior to administration to a patient.

### Methods of Obtaining Reproductive Tissue-Derived Cells

Described below are illustrative methods for processing reproductive tissue to optimize cell yield and viability for clinical applications. In particular, optimized methods for obtaining cell compositions from reproductive tissues obtained during spay or neuter procedures are described. The viable cell compositions obtained by the methods described herein are suitable for clinical applications.

As conventionally understood, neutering is a surgical procedure in which testes and vas deferens are removed from a male (castration). As conventionally understood, spaying is a surgical procedure in which ovaries and fallopian tubes and/or the uterus are removed from a female (ovariohysterectomy).

Associated ancillary tissues including fallopian tubes, epididymis, vas deferens and adipose tissue may also be removed during these procedures.

The below are exemplary Work Instructions:

Dissociation Enzyme Preparation. An enzyme is used to dissociate cells from extracellular matrix and each other, thereby generating a single cell population. In some embodiments, the enzyme used to dissociate cells may include collagenase, chymotrypsin, trypsin, elastase, hyaluronidase, lysozyme, papain, pectinase, pepsin, or any combination thereof. In certain embodiments, a neutral protease (e.g. Dispase) may be combined to improve efficiency.

Tissue processing. Ensure that the biological safety cabinet (BSC) has been prepared with the proper amount of supplies to process the tissue sample prior to processing. Transfer the whole tissue (e.g. from a spay or neuter procedure) into a pre-weighed sterile container of appropriate size. Obtain the mass measurement of the whole tissue. Obtain a new specimen container and carefully transfer the tissue sample into the new specimen container using the sterile forceps. Add between 50-60 mL of PBS to the sterile container containing the tissue. Secure the lid of the sterile container closed and proceed to shake the container to wash excess debris and/or blood from the tissue. Using the sterile forceps and petri dish, carefully transfer the tissue to the inside of the petri dish. Slide the tissue against the interior of the specimen container to allow the excess fluid to drain off the tissue.

Dissect the tissue by obtaining the appropriate region of the reproductive tissue. For tissue isolated from males, using the sterile forceps and small dissecting scissors, excise off and discard the vas deferens and outer epididymis. For tissue isolated from females, using the sterile forceps and small dissecting scissors, excise off and discard the fallopian tubes from the ovaries. For reproductive tissue samples from female subjects that also include the uterus and/or adipose tissue, the uterus and/or adipose tissue may be separately retained for additional processing to ensure enough vascularized tissue mass in the final cell preparations. In all cases, cut the desired excised reproductive tissue sample (including testes or ovaries/uterus) into smaller manageable pieces. Transfer the pieces of tissue into a pre-weighed labelled 50 ml conical tube. Transfer a tissue volume of no more than 22.5 mL measured approximately by markings on the conical tube. Obtain the mass of the reproductive tissue. Proceed to mince the reproductive tissue to approximately 2-3 mm sized pieces. Obtain the dissociation enzyme stock solution. Obtain the mass measurement of minced tissue. An equal volume of enzyme stock solution/PBS shall be added to the observed mass of minced tissue. Calculate the amount of enzyme stock solution and PBS to add to the sample as follows: For every 20 grams of tissue, 1 mL of enzyme stock solution shall be added. Add the appropriate amount of enzyme stock solution and PBS to the minced tissue sample. Secure the lid of the 50 mL conical tube and invert the sample several times to ensure that the tissue sample is mixed.

Tissue Digestion. Verify the dry incubator temperature is between 36-40° C. by observing the temperature of the verification thermometer. Place sample into the incubator block and set the agitation speed to 450+/−50 rpm. The tissue sample shall be incubated for 40+/−5 minutes. After incubation, remove the tissue sample from the incubator. Obtain culture media and aseptically transfer to quench the digestion process.

Tissue Washes. After the addition of culture media to the sample tube, bring the volume of the sample tube to 50 mL with PBS. Mix the sample by inversion. Place the mixed sample tube into a centrifuge set to a speed of 1.400+/−100 rpm at ambient temperature (18-25° C.) with low brake for 15 minutes. Retrieve the sample tube after the centrifugation has completed and aseptically transfer the sample tube into the BSC. Using a sterile conical tube or sterile container, pour off the supernatant. If the pellet remains intact, discard the supernatant into the waste container and proceed. If the pellet is loose, transfer the supernatant back to the original sample tube and centrifuge once again. Add 5-10 mL of PBS to the pellet and resuspend by serological pipet. Obtain a pre-labelled 50 mL conical tube and proceed to cell strain the resuspended cell pellet by one of the following methods:

Non-stacking Cell Strainers. Carefully open the packaging of a 100 μm cell strainer to ensure that the filter portion remains sterile. Set the 100 μm cell strainer onto the pre-labelled 50 ml conical tube. Using a serological pipette, resuspend the pellet and transfer resuspension to the top of the filter. Allow the fluid to drain through the filter. Use the following steps to aid in the filtration of the cell suspension: Using the tip of the serological pipette, mix the contents of the cell strainer to aid in the filtration of the resuspension. Add additional PBS to the top of the filter and mix the contents to allow the filtration. Gently pick up the cell strainer in an upward motion and set it back onto the conical tube to release an air lock which may have occurred.

Rinse the original sample tube with 5-10 mL of PBS and process through the cell strainer. Once the resuspension has completed filtration, remove and dispose of the cell strainer. Set the first filtration conical tube to the side and prepare the next filtration tube. Obtain a new pre-labelled 50 ml conical tube. Carefully open the 70 μm filter to ensure that the filter portion remains sterile. Set the 70 μm cell strainer onto one of the new pre-labelled conical tubes. Using a new serological pipette transfer the cell suspension to the top of the 70 μm filter. Allow the cell suspension to drain through the filter. Rinse the previous tube with 5 mL of PBS and transfer the rinsate to the top of the 70 μm filter. Set to the side and prepare the next filtration tube. Obtain another pre-labelled 50 mL conical tube. Carefully open a 40 μm filter to ensure that the filter portion remains sterile. Set the 40 μm cell strainer onto the new pre-labelled conical tube. Using a new serological pipette transfer the cell suspension to the top of the 40 μm filter. Allow the cell suspension to drain through the filter. Rinse the previous tube with 5 mL of PBS and transfer the rinsate into the 40 μm filter.

Stackable Cell Strainers. Carefully open the packaging of each filter, ensuring to maintain the sterility of the filters. Set the 40 μm filter onto the pre-labelled 50 ml conical tube. Aseptically detach the funnel portion of the remaining two filters. Handle the outside of the filter portion only. Do not touch the filter mesh. Proceed to stack the 70 μm filter on top of the 40 μm filter. Once the 70 μm filter is secured onto the 40 μm filter, proceed to stack the 100 μm filter on top of the 70 μm filter.

Using a serological pipette, resuspend the pellet and transfer resuspension to the top of the stacked filters. Allow the resuspension to drain through the filter system. Use the serological pipette to mix the contents of the top filter to aid the filtrate to go through the filter. Add additional PBS to the top of the filter and mix the contents to allow the filtration. Add 5-10 mL of PBS to the original sample tube and rinse the sides of the tubes by serological pipette. Add the rinsate to the top of the filter and allow to drain through the filter system. It may be necessary to use additional cell strainers should the filter or filter system get clogged. If this is the case, transfer the contents of the filter back into the sample tube and obtain a new filter. Repeat the steps of filtration as instructed above.

After filtering the resuspension either by consecutive cell strainers or stacked cell strainers, add a volume of PBS to bring the volume of the sample tube containing the filtered cell suspension between 35-40 mL and lid the sample tube. Mix by inversion. Transfer the sample tube to the centrifuge and centrifuge the sample at 1,400+/−100 rpm at ambient temperature (18-25° C.) with low brake for 7 minutes. Retrieve the sample tube after centrifuge wash and aseptically transfer into the BSC.

Using a sterile conical tube or sterile container, pour off the supernatant. If the pellet remains intact, discard the supernatant into the waste container and proceed. If the pellet is loose, transfer the supernatant back to the original sample tube and centrifuge once again. Gently flick the pellet to resuspend and bring the sample tube to a volume of 35-40 mL with PBS. Lid the sample tube and mix by inversion. Transfer the sample tube to the centrifuge and centrifuge the sample at 1,400+/−100 rpm at ambient temperature (18-25° C.) with low brake for 7 minutes.

Final Resuspension and Preparation for Cell Counting: While the sample is in the final centrifugation wash step, prepare the BSC with additional serological pipettes and pipet tips and micropipettor. Label microcentrifuge tubes with the sample's unique identifier. Depending on the appearance of the sample (i.e. appears to have a high RBC content), the cell count sampling may need to be diluted. Place the prepared counting sample to the side. Retrieve the sample tube from the centrifuge and aseptically transfer to the BSC. Using a sterile conical tube or sterile container pour off the supernatant. If the pellet remains intact, discard the supernatant into the waste container and proceed If the pellet is loose, transfer the supernatant back to the original sample tube and centrifuge once again.

Fluidize the pellet by gently flicking the pellet. Add a volume of PBS to bring the volume to approximately 2.0 mL. If the cell pellet is large, it may be necessary to bring the volume up to a higher volume. Resuspend the cell suspension gently by pipette and measure the final volume.

Using the micropipettor, obtain a cell count sampling of the cell suspension and place into the prepared sampling tube. Store the cell suspension at 2-8° C. while performing the cell count. Perform cell counting.

Cell Culture Media Preparation Add 445 mL of DMEM with 4.5 g/L glucose, 1× Glutamax and sodium pyruvate to a 500 mL, 0.2 micron, media filtration system. Add 50 mL of EquaFetal serum and 5 mL of penicillin/streptomycin to the filtration system.

Cell Culture. If cell number is below the minimum amount required to achieve an appropriate number of doses, cells may be placed into cell culture to generate culture expanded autologous cell doses.

Resuspended cells were diluted with media to achieve a plating density with the range of 15,000 to 50,000 cells/cm² onto a sterile T75 flask surface. Cells were incubated in a humidified chamber with 5% CO₂ at 37° C. for 6-10 days until cells become 80-90% confluent. Media was replaced after 3 days and again after another 3-4 days as required. This is referred to as the primary (P=0) culture. Upon reaching 80-90% confluence, media was removed, and adherent cells were washed 1× with approximately 10 mL of PBS. PBS was aspirated from the flask. 7 mL of TrypLE was added to the flask, and the flask was incubated for 5-8 minutes at 37° C. in a humidified chamber with 5% CO₂. An equal volume of growth media was added to quench TrypLE activity. Flask was tapped solidly against the palm of a hand to dislodge cells from flask surface. Cells were transferred using either a 10 mL or 25 mL pipette to a 50 mL conical tube containing an additional 15 mL of growth media. Cells were centrifuged for 5-7 minutes at 1350 to 1500 rpm at ambient temperature. After centrifugation, media was removed by either pouring off or aspiration. In either case, care was taken to avoid losing the cell pellet. Pellet was flicked pellet to resuspend the cells and then 30 mL of growth media was added to the cells. Added 15 mL of cell suspension to each of two T175 flasks. Added an additional 15 mL of medium to each flask. Placed flasks of cells into 37° C. humidified chamber with 5% CO₂ and allowed the cells to adhere and proliferate.

Changed media every 3-4 days until cells reach 80-90% confluence. When cells reached 80-90% confluence, media was removed, and adherent cells were washed 1× with approximately 10 mL of PBS. PBS was aspirated from the flask. 15 mL of TrypLE was added to the flask, and the flask was incubated for 5-8 minutes at 37° C. in a humidified chamber with 5% CO₂. An equal volume of growth media was added to quench TrypLE activity. Flask was tapped solidly against the palm of a hand to dislodge cells from flask surface. Cells were transferred using either a 10 mL or 25 mL pipette to a 50 mL conical tube containing an additional 15 mL of growth media.

Cells were centrifuged for 5-7 minutes at 1350 to 1500 rpm at ambient temperature.

After centrifugation, media was removed by either pouring off or aspiration. In either case, care was taken to avoid losing the cell pellet. Pellet was flicked to resuspend the cells into approximately 2.5 to 3.0 mL. 200 μl was removed for cell counting and viability determination. After counting, remaining cells were diluted into ˜17 additional mL of media and centrifuged for 5-7 minutes at 1350 to 1500 rpm at ambient temperature. After centrifugation, media was aspirated, pellet resuspended by flicking and cells were reconstituted into either 2 to 3 mL final volume with cryopreservation media and transferred to prelabeled cryovials and frozen as described herein.

### Media Additives for Culture of Cells

Growth/Tropic Factors—EGF, bFGF, TGF-B1, or -B3, IGF-I, ITS (Insulin, thyroxin, Selenium), non-essential amino acid supplementation, Serum free media (Nucleus Biologics, Physiologix SF)

Priming additives: For bone repair: ascorbate, Dexamethasone, vitamin D3, with or without BMP-2. For cartilage: BMP-6, TGF-B3, ITS. For nerve indications: FGF-10, BDNF, N2 media supplement (ThermoFisher), Conditioned medium from other cell types (i.e. neuronal conditioned media from neuroglial cell/stem cell cultures).

For immunomodulatory indication: Conditioned media from lymphocytes stimulated with proinflammatory agonist (lipopolysaccharide, aka LPS, concanavalin A). Lymphocytes from allogeneic or autologous sources may be used.

For Angiogenesis: decellularized matrix from tissue, VEGF+placental growth factor (PIGF)+Angiogenin or angiopoietin A. Decellularized matrices from allogeneic or autologous sources may be used.

To thaw cells, warm aqueous medium of choice in a 37° C. water bath. Quickly thaw cells in a 37° C. water bath by gently shaking the vial. Allow to thaw for 1.5 to 2 min. Do not submerge the vial. Remove the vial when only a small frozen cell pellet remains. Do not vortex cells. Dilute cells with appropriate amount of warmed medium. Centrifuge the sample at 1450±50 rpm for 5 minutes at room temperature. Remove the supernatant from the tube and resuspend in 2-5 mL of new growth media. Determine cell viability and number.

### Optimized, Alternative Methods of Processing Reproductive Tissues

A client's pet's spay or neuter tissue is collected at a veterinary clinic and shipped in a special container to the Gallant Laboratory. The tissue is processed according to a standardized receiving/in-take protocol. Once the tissue is accepted for processing, it is transferred to the laboratory to initiate the generation of the Primary Cell Fraction (PCF) doses, which is conducted in a sterile manner by using a Biological Safety Cabinet (BSC).

The tissue is minced into small pieces while in the BSC. After mincing, an enzymatic solution is added to digest the tissue pieces. The enzymatic solution is composed of Collagenase and a neutral protease preparation commercially available (e.g., DE10, VitaCyte).

The tissue is kept in suspension on a rocker platform located in a dry heat incubator set at 37° C. for 40 minutes. Once the sample has been digested for 40 minutes, it is returned to the BSC. The reaction is stopped with culture medium containing Fetal Bovine Serum (10%, FBS), followed by dilution with Phosphate Buffered Saline (PBS).

The tissue digest suspension is passed through a stack of cell strainers (300 or 100 μm/70 μm/40 μm stacked strainers (Corning)) to separate the released cells from the tissue pieces. The retained pieces in the filters are washed with PBS, after which the cell strainers are discarded. The collected cells are centrifuged, and washed with PBS twice. Before the second wash, an aliquot is obtained and a cell count and viability is determined. Following the second centrifugation, the supernatant is removed and the cell pellet is resuspended in the appropriate volume of cryopreservation fluid to generate a PCF dose.

To optimize the methods of processing reproductive tissues to obtain reproductive tissue derived cells described herein, the present invention comprises: obtaining reproductive tissues from spay or neuter procedures; mincing said reproductive tissue; digesting said minced reproductive tissue with an enzymatic solution to obtain a tissue suspension; agitating said tissue suspension such that reciprocal mechanical agitation (e.g., rocking) is applied to the tissue suspension; filtering the tissue suspension into a single cell suspension fraction and a partially digested reproductive tissue fraction; centrifuging the cell suspension fraction to form a first centrifuged cell pellet and resuspending the first centrifuged cell pellet into a single cell suspension; incubating the partially digested reproductive tissue fraction in an incubation medium (e.g. at 37° C.) such that cells migrate from the partially digested reproductive tissue fraction into the incubation medium; centrifuging the incubation medium to form a second centrifuged cell pellet and resuspending the second centrifuged cell pellet into a migrating cell suspension; combining the single cell suspension and the migrating cell suspension to obtain the reproductive tissue derived cells.

To optimize the reproductive tissue processing methods of the present invention, use of an enhanced manual mechanical processing technique may be utilized. Specifically, the application of greater mechanical agitation may be used. For example, in an optimized manual method, the tubes containing the tissue pieces and the enzymatic digestion solution may be shaken by hand (40 shakes) every 10 minutes for the duration of the incubation period at 37° C.

In a series of canine uterine tissues processed by the non-optimized method described above with the standard enzyme digestion solution and rocking, the average yield of viable cells per gram of spay tissue processed was 1.3×10⁶ cells/g (n=6). For canine uterine tissues processed with the enhanced manual mechanical agitation (40 shakes every 10 minutes) and standard enzyme digestion fluid protocol outlined herein, the average yield of viable cells per gram of spay tissue processed was 7.0×10⁶ cells/g (n=5).

To further optimize the reproductive tissue processing methods of the present invention, use of an enhanced non-manual mechanical processing technique may be utilized. For example, mechanical agitation can be applied with the use of a rotator, like the PTR-60 Rotator (Grant USA, Inc., Beaver Falls, PA). The PTR-60 provides mechanical agitation in three modes: rotation about the platform's long axis, reciprocal motion, and vibration of the platform. These motions can be programmed, such that less rotational and more reciprocal/vibrational mechanical agitation can be applied. Additional settings include the angle of the platform/duration of the vibration, as well as the arc of reciprocal motion and its duration. These three modes of mechanical agitation are integrated into a single process, which can last for a specified duration. Use of the PTR-60 Rotator with an enzymatic preparation composed of Collagenase MA enzyme material and Thermolysin (both from VitaCyte, Inc.) resulted in an average cell yield/g of spay tissue of 5.54×10⁶ (n=5) and for neuter tissue of 7.16×10⁶ (n=5). Both of these results are substantially above the values obtained from client owned samples processed with the Standard Method (rocking with standard enzymatic digestion solution), in which spay tissue had an average cell yield/g of 1.3×10⁶ (n=6) and neuter tissue had 1.8×10⁶ (n=6).

To optimize the reproductive tissue processing methods of the present invention, modified handling of the partially digested tissue pieces and the cell suspension can be implemented. In typical protocols, the tissue digest is placed in a set of cell strainers in order to separate out the single cells present in the tissue digest from the tissue pieces, after which the cell strainers are discarded. In the optimized protocols of the present invention, after separating the single cell suspension from the tissue pieces, the one or more of the cell strainers containing tissue pieces are placed in a small volume of incubation medium (e.g., PBS) in a sterile 6-well plate and allowed to incubate at 37° C. for 1-2 hours. At the end of the additional incubation period, the tissue pieces are rinsed with PBS to recover additional cells that are associated with the tissue pieces, and the fluid in the well is collected. The collected fluid is centrifuged, the supernatant is discarded and the pellet is resuspended with a small volume of PBS. This preparation is combined with the single cell suspension obtained earlier in the process, which has been stored at 4° C. in the interim. A cell count and viability are determined on the combined aliquots and the preparation is centrifuged. The supernatant removed and the pellet resuspended in the appropriate volume to produce one or more clinical dose. Cell migration over time from partially digested tissue pieces has been demonstrated (Table 1), so the inventive method is intended to recover any additional cells released during the limited duration of the incubation of tissue pieces in PBS.

To further optimize the present invention, partially digested reproductive tissue may be cryopreserved. In the enhanced mechanical agitation method, the tissue pieces are treated with an enzymatic solution that digests a portion of the tissue extracellular matrix thereby releasing cells. However, the degree of digestion will vary depending on properties of the tissue pieces, such as physical dimensions and density of the tissue being digested.

Once the single cell suspension has been recovered, including after an extended resting period of the tissue pieces in a 6-well plate, the tissue pieces can be recovered and suspended in a cryopreservation fluid in a cryovial and placed in LN₂ storage, similar to that used for the clinical doses described above. The benefit of cryopreserving partially digested tissue pieces depends on the presence of viable cells.

In some embodiments, the methods disclosed herein result in an improved total yield of single cells isolated from the reproductive tissue. In some embodiments, combining the single cell suspension and the migrating cell suspension to obtain the reproductive tissue derived cells improves the total yield of single cells isolated from the reproductive tissue. In some embodiments, combining the single cell suspension and the migrating cell suspension to obtain the reproductive tissue derived cells improves the total yield of single cells isolated from the reproductive tissue by at least 50%, 100%, 120%, 140%, 160%, 180%, 200%, 220%, 240%, 260%, 280%, 300%, 320%, 340%, 360%, 380%, or 400% as compared to the total yield of single cells isolated in the cell suspension fraction alone, or any percentage within a range defined by any two of the aforementioned percentages. In some embodiments, combining the single cell suspension and the migrating cell suspension to obtain the reproductive tissue derived cells results in a total yield of single cells isolated from the reproductive tissue of about 3×10\hat{ }6, 4×10\hat{ }6, 5×10\hat{ }6, 6×10\hat{ }6, 7×10\hat{ }6, 8×10\hat{ }6, 9×10\hat{ }6, or 10\hat{ }7 cells per gram of reproductive tissue, or any number of cells within a range defined by any two of the aforementioned number of cells.

### Mechanisms of Action of Reproductive Tissue Derived Cells

In some embodiments, reproductive tissue derived cell functions include anti-inflammatory/immunomodulation effects. The reproductive tissue derived cells obtained using the methods of the invention limit inflammatory responses and promote anti-inflammatory pathways. When present in an inflammatory environment, the cells of the invention may alter the cytokine secretion profile of immune cells causing a shift from a pro-inflammatory environment to an anti-inflammatory or tolerant environment.

In some embodiments, reproductive tissue derived cell functions include trophic support. Reproductive tissue derived cells of the present invention secrete bioactive levels of cytokines and growth factors that support angiogenesis, tissue remodeling, differentiation, and antiapoptotic events. The cells of the present invention secrete a number of angiogenesis-related cytokines such as Vascular Endothelial Growth Factor (VEGF) and Transforming Growth Factor Beta 1 (TGF-β1).

In some embodiments, reproductive tissue derived cell functions include differentiation. Reproductive tissue derived cells of the present invention are expected to demonstrate a diverse plasticity, including differentiation into adipo-, osteo-, chondro-, myo-, cardiomyo-, endothelial, hepato-, neuro-, epithelial, and hematopoietic lineages. Accordingly, the cells of the present invention can repair damaged or diseased tissue via transplant engraftment and differentiation, including, for example, for Achilles tendon repair, digital flexor tendon injury, repairing meniscal injury in osteoarthritis and bone regeneration and repair.

In some embodiments, reproductive tissue derived cell functions exhibit homing. The reproductive tissue derived cells of the present invention are involved in homing or chemotaxis wherein a cell migrates from one area of the body to a distant site of injury where its therapeutic effect is needed. This function results from the secretion of cytokines, such as Monocyte Chemoattractant Protein-1 (MCP-1).

In some embodiments, reproductive tissue derived cell functions include tissue repair and revascularization. The reproductive tissue derived cells of the present invention may be comprised of endothelial progenitor cells, stem cells as well as other progenitor cells that assist in angiogenesis and neovascularization by the secretion of cytokines, such as Platelet Derived Growth Factor-BB (PDGF-BB), Hepatic Growth Factor (HGF), Vascular Endothelial Growth Factor (VEGF), Transforming Growth Factor Beta 1 (TGF-β1), and Macrophage Inflammatory Protein-1β.

In some embodiments, reproductive tissue derived cell functions include anti-apoptosis. Apoptosis is defined as a programmed cell death or “cell suicide”, an event that is genetically controlled. The reproductive tissue derived cells of the present invention reduce cell apoptosis by expressing factors that support cell survival, such as Vascular Endothelial Growth Factor (VEGF), Transforming Growth Factor Beta 1 (TGF-β1) and Hepatocyte Growth Factor (HGF).

### Cryopreservation & Thawing of Reproductive Tissue Derived Cells

In some embodiments, the reproductive tissue derived cell composition are combined with an aqueous cryopreservation medium that includes dimethylsulfoxide (DMSO) in a manner to maintain selected levels of viable cell compositions. DMSO is a chemical used in cryopreservation to create transient holes in cell membranes enabling rapid intermembrane passage of molecules, and has been shown to reduce cytotoxicity during cryopreservation. Alternatives to DMSO that also reduce cytotoxicity during cryopreservation, e.g., DMSO-free cryopreservation media, are also contemplated by the present invention. The reproductive tissue derived cell composition comprising DMSO, may be further combined with an anti-apoptotic agent. The combination of the cells with the DMSO can be conducted in any suitable fashion. The combination of the cells with the DMSO and an anti-apoptotic agent can be conducted in any suitable fashion, including by combining the viable cells with a solution containing both DMSO and an anti-apoptotic agent, or by combining the cells with separate solutions, one of which contains the DMSO and another of which contains the anti-apoptotic agent. It is understood that the cryopreservation medium can contain agents other than the DMSO and an anti-apoptotic agent, including other cryopreservation agents. However, in preferred forms, the DMSO will constitute at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% of the cryopreservation medium other than the water, or any percentage within a range defined by any two of the aforementioned percentages.

In preferred prepared reproductive tissue derived viable cell compositions to be cryopreserved, the cryopreservation medium will include the DMSO at a concentration of about 10% or less, 5% or less or about 3% or less, in the range of about 1% to 3% or in the range of about 1.5% to about 2.5%. In certain preferred forms, the cryopreservation medium includes DMSO at a concentration of about 10%.

In preferred prepared reproductive tissue derived viable cell compositions to be cryopreserved, the viable cells will be present at a concentration of at least about 200,000 cells/ml, at least about 500,000 cells/ml, at least about 1 million cells/ml, at least about 2 million cells/ml, at least about 5 million cells/ml, or at least about 10 million cells/ml, or any concentration of cells within a range defined by any two of the aforementioned concentration of cells.

The reproductive tissue derived viable cell compositions obtained using the methods of the invention demonstrate a high degree of viability, both before and after storage at 4° C. or under liquid nitrogen, and after being shipped at temperatures less than 12° C. or on ice. In certain embodiments, the percentage of viable cells, as determined by standard counting methods, e.g., Trypan blue dye exclusion methods, immediately following preparation of a viable cell composition of the present invention is at least 30%, 40%, 50%, 60%, 65%, 70%, 80%, 90% or 95%. In related embodiments, the percentage of viable cells, as determined by standard counting methods, e.g., Trypan blue dye exclusion methods, followed by thawing is at least 30%, 40%, 45%, 50%, 60%, 70%, 80%, 85% or 90%. In another embodiment, the percentage of viable cells, as determined by standard counting methods, following refrigerated storage or shipment on ice packs for less than 24 hours is at least 30%, 40%, 50%, 60%, 70%, 80% or 90%.

The reproductive tissue derived viable cell composition including the viable cells and the above-disclosed cryopreservation medium, in unfrozen (liquid) form, can be prepared in, delivered into, or otherwise provided in a cryopreservation container such as a cryopreservation vial or cryopreservation bag. In preferred forms, each cryopreservation container will contain about 1 ml to about 10 ml of the cell composition, more preferably about 1 ml to about 5 ml of the composition, and most preferably about 1 ml to about 3 ml of the composition. Preferred cryopreservation vials for use in embodiments herein are commercially available.

The cryopreservation container containing the viable cell composition can be subjected to cryopreservation conditions to cause the composition to be cryopreserved. For example, the cryopreservation conditions can include storing the cryopreservation container at a temperature at which the viable cell composition freezes. For example, the cell composition can be maintained in a cryopreserved state at a temperature in the range of about −60° C. or lower, for example in the range of about −60° C. to −150° C., more preferably about −60° C. to −100° C., and even more preferably about −70° C. to −90° C. In certain forms, the cell composition can be maintained in a cryopreserved state at a temperature of about −80° C./−5° C. In some embodiments, the cryopreservation container containing the viable cell composition can be stored under cryopreservation conditions (e.g. immersed in liquid nitrogen and/or in a mechanical freezer) at a surgical facility, a manufacturing facility, a storage facility, or a distribution facility, or any other facility near the point-of-care facility, the cryopreservation container containing the viable cells can be shipped to the point of care while maintaining cryopreservation conditions (e.g. in a shipping package containing a liquid nitrogen system and/or dry ice/frozen carbon dioxide), and the cryopreservation container containing the viable cells can be stored at the point of care (e.g. a veterinary clinic or hospital) under cryopreservation conditions until needed for administration, preferably within a mechanical freezer as disclosed herein.

Preferred reproductive tissue derived viable cell compositions containing the cells and the aqueous cryopreservation medium (e.g. those comprising DMSO) exhibit good capacity to maintain the viability of the cells during storage at a temperature in the range of about −60° C. to −196° C. For example, depending on the composition of the initial patient-derived samples, preferred compositions will lose no more than 20% of their initial viable cells, no more than 10% of their viable cells, or no more than 5% of their viable cells, when stored at a temperature in the range of −60° C. to −196° C. for a period of six months, and more preferably when stored at a temperature in the range of −60° C. to −196° C. for a period of one year. Such storage stabilities under the relatively moderate cooling conditions enable advantageous product distribution and use methods in which multiple point of care locations (e.g. 10 or more, or 20 or more locations) can maintain mechanical freezers in which a plurality of cryopreservation containers each containing a cell composition as described herein are stored. The stability of the viability of the cells under these storage conditions can provide an acceptable shelf life at the point of care. In some embodiments, the viability of the cells remain stable or close to stable for hours (e.g. within 1, 2, 3, 4, 5, 6, 7, 8, 9, 10, 11, 12, 13, 14, 15, 16, 17, 18, 19, 20, 21, 22, 23, or 24 hours). Immediately prior to administration (e.g. within 6 hours, or within 2 hours, or within 1 hour), a cryopreservation container can be removed from the freezer and the composition therein thawed for additional manipulation (e.g. dilution as described below) and/or administration.

In some methods herein, to thaw the cell composition, the cryopreservation container can be caused to warm to a temperature at which the frozen cell composition reverts to a liquid form. This can be achieved in any suitable manner. For example, the container, remaining sterilely sealed, can be exposed to a gaseous environment (e.g. the room air environment at the point of care) at room temperature (e.g. about 20° C. to 25° C.) to thaw the cell composition. In another form, the container can be incubated in a heated liquid or bead bath to thaw the cell composition, for example heated to a temperature in the range of about 33° C. to 37° C., preferably about 37° C. The cryopreservation and subsequent thawing of the cell composition will preferably result in high maintenance of cell viability, for example with the thawed composition enabling recovery of at least 60% to at least 90% viable cells.

After thawing, the viable cell composition can be removed from the cryopreservation container. For example, in some embodiments, the cryopreservation container has a septum through which a needle or other cannulated device may be passed to access the container contents, while maintaining a sterile environment within the container. The needle or other cannulated device can then be used to withdraw the cell composition from the container sterilely, for example into a syringe barrel or other vessel. It will be understood that other methods for accessing the cell composition within and removing the viable cell composition from the cryopreservation container may be used in other embodiments.

In some forms, the viable cell composition can be administered to a patient, or put to a research or other use, unmodified from its composition upon removal from the cryopreservation container. In some embodiments, the viable cell composition is modified for use, including for use in administration to a patient. The modification can include, for example, the addition of one or more substances to the viable cell composition.

In preferred methods, the viable cell composition is combined with an aqueous medium to dilute the cell concentration of the composition, and potentially also dilute the concentration of other components, such as DMSO, of the viable cell composition. In beneficial aspects, the aqueous medium can be a physiologically acceptable aqueous liquid, such as a phosphate-buffered or otherwise buffered saline solution, potentially with other additives. Also, it will be understood that the aqueous medium can be combined with the viable cell composition as a single volume of a given composition, or as multiple volumes of the same composition or of differing compositions.

In preferred methods, to prepare a diluted viable cell composition for administration to a patient or other use, an aqueous medium will be combined with the cell composition in a volume ratio of at least 2:1, at least 3:1, at least 5:1, or at least 8:1. In more preferred forms, such volume ratio will be in the range of about 3:1 to about 20:1, or about 5:1 to about 15:1. It is contemplated that the cell concentration and DMSO concentration in the prepared diluted composition will be correspondingly reduced relative to the starting cell composition prior to dilution; however, in some forms, the aqueous medium used to dilute may itself contain some concentration of cells, additives, agents and/or DMSO, albeit typically lower than the concentration in the starting cell composition so as to result in some dilution of cells, and DMSO in the diluted cell composition.

The combination of the thawed cell composition with the aqueous medium can be conducted in any suitable container or vessel. In beneficial modes, the combination is conducted in a second container (other than the cryopreservation or other container in which the cells were stored or held). This second container can include an input port or other input member, for example a septum, for sterile transfer of materials such as the cell composition and/or the aqueous medium into the second container. In some forms, combining the cell composition and aqueous medium can include delivering both of these into the second container, for example in either order or simultaneously. In other forms, the second container can be provided as a pre-manufactured container already containing the aqueous medium in sterile condition, and the cell composition can be added to the pre-manufactured container. In any of these embodiments, the second container can be a bag and/or can have an outlet port spaced from the input port or other member for delivery of the prepared diluted cell composition from the bag or other container, e.g. for delivery into a patient. The second container can be a bag having a septum for sterile input of materials and a valved port for outlet of materials, e.g. as occurs in common saline bags for patient treatment in medical care. The thawed cell composition, and potentially the diluting medium (if not pre-manufactured in the bag) can be sterilely delivered into the bag by needle through the septum, and the prepared diluted cell composition can be sterilely delivered to a patient through the valved port.

In certain embodiments, the aqueous medium will be combined with the viable cell composition, and the prepared diluted cell composition will have a DMSO concentration that has been reduced from its initial level (e.g. any of those levels indicated above for the cryopreserved cell composition) to a diluted concentration of no more than about 5%, 4%, 3%. 2%, 1%, 0.5%, 0.3%, 0.1%, or 0.05% or any concentration of DMSO within a range defined by any two of the aforementioned concentration. In some embodiments, the concentration of DMSO is about 0.3% or less. In some embodiments, the concentration of DMSO is in a range of about 0.05% to about 0.5% or in a range of about 0.1% to about 0.3%. In regard to the cell concentration in the diluted cell composition, the aqueous medium will typically not contain any cells, and the cell concentration of the diluted viable cell composition will the lower than that of the starting cell composition, for example in the range of about 100,000 cells/ml to about 20 million cells/ml, or about 250,000 cells/ml to about 5 million cells/ml, or about 500,000 cells/ml to about 2 million cells/ml. In certain forms, the cell concentration of the prepared diluted cell composition will be about 1 million cells/ml.

In certain embodiments herein, the aqueous medium will contain an agent or additive to reduce cell death post-thaw, e.g., anti-apoptotic agent, additive or compound. Examples of anti-apoptotic agents or additives include, for example, Anti-Thrombospondin antibodies, compounds that promote expression of Bcl-2 and cell survival, inhibitors of radical oxygen species production, including but not limited to, vitamin E, melatonin, resveratrol, carnosine, coenzyme Q10, Caspase Inhibitors, GSK-3B Inhibitors: Lithium, paullone, indirubines, thiazoles, aminopyrimidines, bisindol-malcimides (SB-216763 and SB-41528, which were developed by GlaxoSmithKline), PPAR agonists (e.g. rosiglitazone and troglitazone), MinoCycline, Cox-2 Inhibitors, or Celecoxib. Additional protective aqueous compositions may include commercial products, like Cell Thawing Medium (BioLife Solutions) or HypoThermosol (BioLife). Accordingly, the aqueous medium will be combined with the cell composition, and the prepared diluted cell composition will have an anti-apoptotic agent concentration that is greater than that of the DMSO and/or which is at least about 0.1%, more preferably at least about 0.3%, and in some embodiments a concentration in the range of about 0.1% to about 6% or in the range of about 0.3% to about 2%.

The aqueous medium can contain any suitable concentration of agents or additives. For example, an anti-clumping additive may be used for these purposes. In certain aspects, the aqueous medium will contain an agent or additive, e.g., an anti-clumping agent or additive, at a concentration of about 1% to about 20% by weight, or about 1% to about 10% by weight, or about 2% to about 7% by weight, or about 2.5% to about 5% by weight, or about 3% to about 4% by weight. The aqueous medium, whether it contains an agent or additive as specified herein or not, can also include other components and/or have a specified osmolarity. For example, it can include sodium chloride at a physiologically acceptable level, for example at a level in the range of about 0.5% to about 1.5%, e.g. about 0.9% (isotonic). The aqueous medium can also include a buffer, for example phosphate buffer, and can have a pH in the range of about 6 to 8, or about 6.8 to 7.8, or about 7 to 7.5. The aqueous medium can also have an osmolarity in the range of 200 to 600 milliosmols per kilogram (mosm/kg), or 250 to 500 mosm/kg, or 250 to 400 mosm/kg.

An aqueous medium containing an agent or additive, e.g., an anti-clumping agent or additive can be combined with the cell composition containing DMSO, to prepare an agent or additive, e.g., an anti-clumping agent or additive-containing diluted cell composition having a suitable concentration of an agent or additive, e.g., an anti-clumping agent or additive, along with DMSO (e.g. with the DMSO in the concentrations for the diluted cell composition specified above). This concentration of an agent or additive, e.g., an anti-clumping agent or additive in the prepared diluted viable cell composition, in certain embodiments, is in the range of about 1% to about 10% by weight an agent or additive, e.g., an anti-clumping agent or additive or about 2% to about 7% by weight, or about 2.5% to about 5% by weight, or about 3% to about 4% by weight. Additionally, or alternatively, the concentration of an agent or additive, e.g., an anti-clumping agent or additive can be effective to inhibit clumping of the cells as compared to a corresponding cell composition without an agent or additive, e.g., an anti-clumping agent or additive. Inhibition of clumping can be observed by the formation of fewer and/or smaller clumps of cells in the prepared cell composition, for example at a time point at least ten minutes after preparation of the cell composition, at least twenty minutes after preparation of the cell composition, or at least after 60 minutes after preparation of the cell composition. The capacity of an agent or additive, e.g., an anti-clumping agent or additive to inhibit clumping for significant periods of time following preparation of the cell composition can, for example, provide sufficient time to administer the prepared cell composition to a patient, for example, by injecting or infusing the cell composition into the bloodstream of a patient by venous or arterial access and/or by local implantation of the cell composition. In therapeutic applications of cell compositions, the composition can be administered to the patient over a relatively prolonged period of time, for example at least 10 minutes, at least 20 minutes, or at least 60 minutes.

As disclosed herein, the prepared diluted viable cell composition can have specified concentrations of cells, DMSO, an agent or additive, e.g., an anti-apoptotic agent or additive and/or an agent or additive, e.g., an anti-clumping agent or additive. Where minimum or maximum concentrations, or ranges of concentrations, are stated, it will be understood that the conditions of the dilution of the starting viable cell composition can be controlled to achieve the stated amounts in the prepared diluted viable cell composition. These conditions include, for example, the concentrations of components in the starting viable cell composition prior to dilution, the volume ratio of the aqueous medium used relative to the starting viable cell composition, and the concentrations (if any) of the identified components in the aqueous medium.

Additional embodiments herein include products useful in preparing diluted cell compositions as described herein. In one embodiment, provided is an aqueous medium useful for preparing a diluted cell composition. The aqueous medium can contain those components, and in amounts, as specified herein. As well, the aqueous medium can be provided in sterile form in a container that is included in a kit. That container may be a vial, bag or other container. In certain forms, the container has the features of the “second container” discussed hereinabove in which the diluted cell composition can be prepared, including for example having an inlet port or other member (e.g. needle septum) and a separate outlet port as discussed above. Kits disclosed herein may include the container containing the aqueous medium along with one or more additional components, for example including but not limited to a liquid transfer device such as a syringe and attached or attachable needle, and potentially also a container containing the starting cell composition to be used to prepare the diluted cell composition. The container containing the starting cell composition can include the cell composition in a cryopreserved state (e.g. shipped frozen with the kit) or in a non-cryopreserved (e.g. thawed where the cells were previously cryopreserved) state, including embodiments in which the cryopreserved cell composition contains DMSO in any of those amounts therefor specified above. Kits disclosed herein may also include at least one filter, for example a Hemo-Nate filter, through which a prepared diluted cell composition can be passed prior to administration into a patient, and/or tubing through which the diluted cell composition can be passed during administration to a patient.

### Cell Delivery

The terms “individual,” “patient.”, “subject” interchangeably refer to a mammal, for example, a human, a non-human primate, a domesticated mammal (e.g., a canine or a feline), an agricultural mammal (e.g., equine, bovine, ovine, porcine), or a laboratory mammal (e.g., rattus, murine, lagomorph, hamster).

As used herein, the terms “treating” and “treatment” refer to delaying the onset of, retarding or reversing the progress of, reducing the severity of, or alleviating or preventing either the disease or condition to which the term applies (i.e., dry eye syndromes or dry eye diseases), or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

The term “effective amount” or “therapeutically effective amount” refers to the amount of an active agent sufficient to induce a desired biological result (e.g., prevention, delay, reduction or inhibition of one or more symptoms). That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, “administering” refers to local and systemic administration, e.g., including enteral and parenteral administration. Routes of administration for the cells that find use in the present invention include, e.g., administration as a suppository, intravenous (“iv”), intraperitoneal (“ip”), intramuscular (“im”), intradermal, intrathecal (“it”), intralesional, intranasal, or subcutaneous (“sc”) administration. Administration may also be local to the target or damaged tissue. For example, for dry eye disorders, the cells may be administered, engrafted or transplanted peri-ocularly, intra-lacrimally, peri-lacrimally, subconjunctivally, into the eyelid stroma, e.g., at the lid margin, into or around the meibomian glands. Administration can be by any appropriate route such that the immunosuppressive agents secreted by the cells prevent, reduce or inhibit destruction or damage to the target tissue, e.g., the lacrimal gland and/or the meibomian glands and/or goblet cells of the conjunctiva. Parenteral administration includes, e.g., intravenous, intramuscular, intra-arteriole, intraventricular, intradermal, intrathecal, subcutaneous, intraperitoneal, and intrarectal administration.

In certain embodiments, the viable cell populations are present within a composition adapted for and suitable for delivery to a patient, i.e., physiologically compatible. Accordingly, compositions of the viable cell populations will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

Cell populations and related compositions may be provided to a patient by a variety of different means. The prepared cell composition can be administered to a patient in any suitable manner. In certain embodiments, they are provided locally, e.g., to a site of actual or potential injury. In one embodiment, they are provided using a syringe to inject the compositions at a site of possible or actual injury or disease. In other embodiments, they are provided systemically. In certain forms, the cell composition is delivered systemically into the bloodstream of a patient, for example by delivery into a vein or artery. In one embodiment, they are administered to the bloodstream intravenously or intra-arterially. The particular route of administration will depend, in large part, upon the location and nature of the disease or injury being treated or prevented. Accordingly, the invention includes providing a cell population or composition of the invention via any known and available method or route, including but not limited to oral, parenteral, intravenous, intra-arterial, intranasal, intradermal, intrathecal, and intramuscular administration. Any of these or any combination of these modes of administration may be used in the treatment of a patient.

In certain combination treatments, a first amount of a prepared cell composition herein can be delivered systemically into the bloodstream of a patient, and a second amount of a prepared cell composition herein (e.g. prepared with or separately from the first amount and including the same type(s) or a different type(s) of cells) is implanted locally in or near one or more skeletal joints in a patient to treat an arthritic condition, e.g. any of those arthritic conditions identified herein. Also, in patient treatments herein, a single administration of a prepared cell composition as described herein can be made in some embodiments, while in others multiple separate administrations of prepared cell compositions as described herein may be made over time (e.g. weekly or monthly administrations). In further embodiments, the prepared, diluted cell composition can be filtered prior to administration to the patient, e.g. to remove any clumps of cells that may be present. In certain forms, the cell composition can be passed through an in-line filter positioned in tubing through which the cell composition is passed into the bloodstream of the patient, e.g. into a vein or artery of the patient. Such a filter can, in certain variants, have a particle size cutoff of about 200 micrometers (i.e. exclude from passage particles having a maximum cross-sectional dimension of greater than about 200 micrometers) or lower, or a particle size cutoff of about 170 micrometers or lower, or a particle size cutoff of about 100 micrometers or lower, while allowing the passage of singly suspended cells through the filter.

In one specific embodiment, a method of treatment comprises injecting a composition comprising viable cells isolated from reproductive tissue of a dog, cat or horse and prepared according to a method of the invention into the same or different dog, cat or horse at a site of actual or potential injury, such as a tendon or ligament.

The development of suitable dosing and treatment regimens for using the cell populations and compositions described herein in a variety of treatment regimens, including e.g., oral, parenteral, intravenous, intranasal, intrathecal, and intramuscular administration and formulation, will again be driven in large part by the disease or injury being treated or prevented and the route of administration. The determination of suitable dosages and treatment regimens may be readily accomplished based upon information generally known in the art.

Treatment may comprise a single treatment or multiple treatments. In particular, for preventative purposes, it is contemplated in certain embodiments that viable cell populations of the invention are administered prior to a stress that might potentially cause injury, such as, e.g., an animal race (e.g., dog or horse race).

### EXAMPLES

### Example 1: Standard Reproductive Tissue Processing Protocols

A client's pet's spay or neuter tissue is collected at a veterinary clinic and shipped in a special container to the Gallant Laboratory. The tissue is processed according to a standardized receiving/in-take protocol. Once the tissue is accepted for processing, it is transferred to the laboratory to initiate the generation of the PCF doses, which is conducted in a sterile manner by using a Biological Safety Cabinet (BSC).

The tissue is minced into small pieces while in the BSC. After mincing, an enzymatic solution is added to digest the tissue pieces. The enzymatic solution is composed of Collagenase and a neutral protease preparation commercially available (DE10, VitaCyte).

The tissue is kept in suspension on a rocker platform located in a dry heat incubator set at 37° C. for 40 minutes. Once the sample has been digested for 40 minutes it is returned to the BSC. The reaction is stopped with culture medium containing Fetal Bovine Serum (10%, FBS), followed by dilution with Phosphate Buffered Saline (PBS).

The tissue digest suspension is passed through a stack of cell strainers (300 or 100 μm/70 μm/40 μm stacked strainers (Corning)) to separate the released cells from the tissue pieces. The retained pieces in the filters are washed with PBS, after which the cell strainers are discarded. The collected cells are centrifuged, and washed with PBS twice.

Before the second wash, an aliquot is obtained and a cell count and viability is determined. Following the second centrifugation, the supernatant is removed and the cell pellet is resuspended in the appropriate volume of cryopreservation fluid to generate a PCF dose.

### Example 2: Optimized Reproductive Tissue Processing Protocols

### Use of an Enhanced Manual Mechanical Processing Technique for Generating PCF

An important change to the Standard protocol listed above involves the application of greater mechanical agitation. In the modified manual method, the tubes containing the tissue pieces and the enzymatic digestion solution are shaken by hand (40 shakes) every 10 minutes for the duration of the incubation period at 37° C. In a series of canine uterine tissues processed by the Standard Protocol with the standard enzyme digestion solution, the average yield of viable cells per gram of spay tissue processed was 1.3×10⁶ cells/g (n=6). For canine uterine tissues processed with the enhanced manual mechanical agitation and standard enzyme digestion fluid protocol outlined in this section, the average yield of viable cells per gram of spay tissue processed was 7.0×10⁶ cells/g (n=5).

### Use of an Enhanced Non-Manual Mechanical Processing Technique for Generating PCF

Mechanical agitation also can be applied with the use of a rotator, like the PTR-60 Rotator (Grant USA, Inc., Beaver Falls, PA). The PTR-60 provides mechanical agitation in three modes: rotation about the platform's long axis, reciprocal motion, and vibration of the platform. These motions can be programmed, such that less rotational and more reciprocal/vibrational mechanical agitation can be applied. Additional settings include the angle of the platform/duration of the vibration, as well as the arc of reciprocal motion and its duration. These three modes of mechanical agitation are integrated into a single process, which can last for a specified duration.

Use of the PTR-60 Rotator with an enzymatic preparation composed of Collagenase MA enzyme material and Thermolysin (both from VitaCyte, Inc.) resulted in an average cell yield/g of spay tissue of 5.54×10⁶ (n=5) and for neuter tissue of 7.16×10⁶ (n=5). Both of these results are substantially above the values obtained from client owned samples processed with the Standard Method, in which spay tissue had an average cell yield/g of 1.3×10⁶ (n=6) and neuter tissue had 1.8×10⁶ (n=6).

Modified Handling of the Tissue Pieces after the Cells are Separated

In the Standard protocol, the tissue digest is placed in a set of cell strainers in order to separate out the single cells present in the tissue digest from the tissue pieces, after which the cell strainers are discarded. In a further modification to the Standard protocol, after separating the single cell suspension from the tissue pieces, the one or more of the cell strainers containing tissue pieces are placed in a small volume of PBS in a sterile 6-well plate and allowed to incubate at 37° C. for 1-2 hours. At the end of the additional incubation period, the tissue pieces are rinsed with PBS to recover additional cells that are associated with the tissue pieces, and the fluid in the well is collected.

The collected fluid is centrifuged, the supernatant is discarded and the pellet is resuspended with a small volume of PBS. This preparation is combined with the single cell suspension obtained earlier in the process, which has been stored at 4° C. in the interim. A cell count and viability are determined on the combined aliquots and the preparation is centrifuged, the supernatant removed and the pellet resuspended in the appropriate volume to produce a PCF dose. Cell migration over time from partially digested tissue pieces has been demonstrated (Table 1), so the inventive method is intended to recover any additional cells released during the limited duration of the incubation of tissue pieces in PBS.

### Cryopreservation of Digested Tissue Pieces

In the enhanced mechanical agitation method, the tissue pieces are treated with an enzymatic solution that digests a portion of the tissue extracellular matrix thereby releasing cells. However, the degree of digestion will vary depending on properties of the tissue pieces, such as physical dimensions and density of the tissue being digested, and between tissues isolated from different subjects. Once the single cell suspension has been recovered, including after an extended resting period of the tissue pieces in a 6-well plate, the tissue pieces can be recovered and suspended in a cryopreservation fluid in a cryovial and placed in LN₂ storage, similar to that used for the PCF doses. The benefit of cryopreserving partially digested tissue pieces depends on the presence of viable cells.

The presence of viable cells in the partially digested tissue pieces obtained after being treated with the enhanced manual agitation method (40 shakes every 10 minutes) described above was observed in an experiment in which the tissue pieces were placed into culture after being separated from the single cell suspension. On Day 1 after plating, the tissue pieces were recovered from the culture flask and transferred to a new culture flask, and the process was repeated on Days 2, 3 and 4. The cells in each flask were recovered by a standard enzymatic detachment method and counted. The total number of cells obtained from two donor uterine tissue-derived digestions with a manual mechanical agitation method are shown in Table 1.

TABLE 1
Total Cell Number Obtained from a Uterine Tissue-
derived Digestion with Manual Mechanical Agitation
Following Culturing of the Tissue Pieces

| Donor ID | Culture Day | Total Cell Number (×10⁵) |
|---|---|---|
| G0310 | D 1 | 6.6 |
| | D 2 | 22 |
| | D 3 | 5.3 |
| | D 4 | 4.8 |
| G0311 | D 1 | 0.3 |
| | D 2 | 0.3 |
| | D 3 | 0.06 |
| | D 4 | 0.07 |

As shown in Table 1, the number of cells that migrate out of the cultured tissue pieces varied with the time in culture and also are donor dependent. However, the partially digested tissue pieces obtained from both donors contain viable cells that are able to migrate out of the tissue pieces over time.

Cryopreservation of Partially Digested Tissue Pieces Obtained from Enzymatic Digestions

Digestion of tissue, like spay and neuter tissue, can be performed to obtain a tissue-free preparation, as described in the section on the Standard Protocol to Produce PCF, which is intended for therapeutic use in the same patient from which the tissue was obtained (autologous use).

In another embodiment, cryopreservation of both partially digested tissue and the released cells is supported by the existence of viable cells in the tissue pieces as demonstrated in Table 1. There are two options for storing the tissue and cells obtained following digestion of tissue. If the PCF preparation is intended to be used as a therapeutic dose, the cryopreserved tissue should be stored separately from the PCF “dose”. In this embodiment, there are PCF doses, and separately, the patient's tissue pieces available. In this arrangement, PCF doses could be supplied upon request. However, if the client/owner of the patient requested that the number of cells be increased, both the PCF preparation and the tissue pieces could be placed into culture. On the other hand, if the PCF preparation will always be cultured in the future, it would be advantageous to store the cells and tissue pieces together in the same container. The preparation where tissue pieces and cells are stored together is called Primary Cell Fraction-Tissue (PCF-T).

This arrangement would be of use if the tissue is being digested to create allogeneic doses. In this situation, the released cells will only be cultured in the future, so there is no need to separate the cells from tissue pieces during cryopreservation. Cryopreservation of the partially digested tissue pieces provides a means to increase the cell number obtained when the PCF and tissue pieces are cultured together, since the tissue pieces are a source of additional viable cells, as shown in Table 1. One option to culture a patient's cells is to thaw the PCF dose and place the recovered cells into tissue culture flasks. If the partially digested tissue pieces were cryopreserved, the cryopreserved tissue could be recovered, and placed in the tissue culture flasks with the thawed cells from the PCF dose. The benefit of this approach is that any cells remaining in the partially digested tissue would respond to the culture medium and migrate out of the tissue pieces. Growth factors present in the tissue also would diffuse out into the culture fluid. The addition of tissue-derived growth factors and cells migrating from the partially digested tissue will increase the probability of successfully expanding the patient's cells, thereby creating more therapeutic doses.

A similar opportunity exists when a PCF preparation is found to be contaminated and is placed into culture. The patient's cryopreserved tissue could be thawed and combined with the PCF cells in order to increase the potential for successful expansion of the PCF cells.

In a situation where the donor tissue is intended to be used for creating allogeneic doses, the storage of cells and partially digested tissue together in a vial provides an opportunity to retain cell/tissue preparations for subsequent use during future culture cycles without the need to obtain and process new donor tissue. This approach allows for management of workload and offers a means of maintaining a reserve source of previously digested tissue and cells.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to.” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The contents of all cited references, including literature references, issued patents, published patent applications, and co-pending patent applications, cited throughout this application are hereby expressly incorporated by reference in their entirety. To the extent that publications and patents or patent applications incorporated by reference contradict the disclosure contained in the specification, the specification is intended to supersede and/or take precedence over any such contradictory material. Those skilled in the art will recognize, or be able to ascertain using no more than routine experimentation, many equivalents to the specific embodiments of the invention described herein. Such equivalents are intended to be encompassed by the appended claims.

1.-32. (canceled)

33. A method of producing a composition comprising reproductive tissue derived cells, the method comprising:
a. mincing isolated mammalian reproductive tissue;
b. mixing the minced tissue with an enzymatic solution to produce a tissue suspension;
c. mechanically agitating the tissue suspension;
d. filtering the mechanically agitated tissue suspension to produce a cell suspension fraction and a partially digested reproductive tissue fraction;
e. centrifuging the cell suspension fraction to form a first centrifuged cell pellet and resuspending the first centrifugal cell pellet into a single cell suspension;
f. incubating the partially digested reproductive tissue fraction in an incubation medium, wherein the cells migrate from the partially digested reproductive tissue fraction into the incubation medium;
g. centrifuging the incubation medium comprising the migrated cells to form a second centrifuged cell pellet and resuspending the second centrifuged cell pellet into a migrating cell suspension; and
h. combining the single cell suspension and the migrating cell suspension to produce the composition comprising reproductive tissue derived cells.

34. The method of claim 33, wherein the reproductive tissue is tissue from a testes, ovary, vas deferens, outer epididymis, fallopian tube, uterus, or any combination thereof.

35. The method of claim 33, wherein the reproductive tissue is human tissue.

36. The method of claim 33, wherein the reproductive tissue is canine tissue or feline tissue.

37. The method of claim 33, wherein the reproductive tissue is obtained from a spay or neuter procedure.

38. The method of claim 33, wherein the enzymatic solution is comprised of collagenase, a neutral protease, or a combination thereof.

39. The method of claim 33, wherein the mechanical agitation is performed for 40, 50, 60, 70, 80, 90, 100, 110, 120, 130, 140, 150, 160, 170, 180, 190, or 200 minutes.

40. The method of claim 39, wherein the mechanical agitation is performed for at least 40 minutes.

41. The method of claim 33, wherein the mechanical agitation is performed for no more than 5, 10, 15, 20, 25, 30, 35, or 40 minutes.

42. The method of claim 41, wherein the mechanical agitation is performed for no more than 40 minutes.

43. The method of claim 33, wherein the mechanical agitation applies reciprocal and vibrational mechanical agitation to the tissue suspension.

44. The method of claim 33, wherein the mechanical agitation does not apply rotational mechanical agitation to the tissue suspension.

45. The method of claim 33, wherein the mechanical agitation is performed on a platform.

46. The method of claim 33, wherein the filtration comprises passing the tissue suspension through a cell strainer to separate the cell suspension fraction and the partially digested tissue fraction.

47. The method of claim 46, wherein the cell strainer comprises a 300 μm cell strainer, 100 μm cell strainer, 70 μm cell strainer, 40 μm cell strainer, or any combination thereof.

48. The method of claim 33, further comprising storing the single cell suspension fraction at 4° C. until combining the single cell suspension with the migrating cell suspension.

49. The method of claim 33, wherein the incubation of the partially digested reproductive tissue fraction is performed for no more than 1, 2, 3, 4, 5, 6, 7, 8, 9, 10, 11, 12, 13, 14, 15, 16, 17, 18, 19, 20, 21, 22, 23, or 24 hours.

50. The method of claim 49, wherein the incubation of the partially digested reproductive tissue fraction is performed for no more than 2 hours.

51. The method of claim 33, wherein the method produces about 3×10⁶ to about 1×10⁷ single cells per gram of reproductive tissue.

52. The method of claim 33, further comprising freezing the composition comprising reproductive tissue derived cells.
